(19) United States
(12) Patent Application Publication
(10) Pub. No.: US 2014/0180057 A1
(43) Pub. Date: Jun. 26, 2014

(54) AORTIC ARTERY MEASURING PROBE, DEVICE AND METHOD OF MEASURING DIAMETER OF AORTIC ARTERY

(71) Applicant: Industrial Technology Research Institute, Hsinchu (TW)

(72) Inventors: Yio-Wha Shau, Taipei City (TW); Hong-Dun Lin, Taipei City (TW)

(73) Assignee: Industrial Technology Research Institute, Hsinchu (TW)

(21) Appl. No.: 14/139,786

(22) Filed: Dec. 23, 2013

Related U.S. Application Data
(60) Provisional application No. 61/745,759, filed on Dec. 24, 2012.

(30) Foreign Application Priority Data
Oct. 25, 2013 (TW) 102138746

(51) Int. Cl. A61B 5/107; A61B 5/02; A61B 5/00

(52) U.S. Cl. CPC A61B 5/1075; A61B 5/0059; A61B 5/1079; A61B 5/02007; A61B 5/742
USPC 600/407

(57) ABSTRACT

An aortic artery measuring probe, device and a method of measuring the diameter of the aortic artery are provided. The aortic artery measuring device includes the aortic artery measuring probe and a signal processing module electrically connected to the aortic artery measuring probe. The aortic artery measuring probe includes a flexible substrate and a sensor array disposed thereon, wherein the sensor array includes M×N ultra-wideband sensors. The ultra-wideband sensors is positioned on a subject and the flexible substrate is deformed to a profile conforming to the profile of the subject. The ultra-wideband sensors transmit a radio wave into the subject and then the radio wave is reflected by a tissue interface of the artery wall of the aortic artery to form a reflected signal. The ultra-wideband sensors receive the reflected signal and the signal processing module analyzes the reflected signal to define the diameter of the aortic artery.

### CROSS-REFERENCE TO RELATED APPLICATION

This application claims the priority benefits of U.S. provisional application Ser. No. 61/745,759, filed on Dec. 24, 2012 and Taiwan application serial no. 102138746, filed on Oct. 25, 2013. The entirety of each of the above-mentioned patent applications is hereby incorporated by reference herein and made a part of this specification.

### BACKGROUND

1. Technical Field

The technical field relates to a measuring probe, a device, and a method of measurement. More particularly, the technical field relates to an aortic artery measuring probe, an aortic artery measuring device, and a method of measuring a diameter of an aortic artery.

1. Related Art

In advanced western countries, aortic aneurysm has become one of the top ten causes of death for people aged 55 and older. In the United States for example, approximately 15,000 people die from the rupture or dissection of an aortic aneurysm each year. In one large-scale research screening, male smokers run a high risk of aortic aneurysm (6.3% with abdominal aortic aneurysm diameter >3 cm). Compared with non-smokers (1.3% with abdominal aortic aneurysm diameter >3 cm), the risk can be as high as five times. For female smokers, only 1.5% have abdominal aortic aneurysm diameter of greater 3 cm.

There are currently three types of measurement devices for measuring the physical conditions of an aortic artery: ultrasound, computed tomography (CT), and nuclear magnetic resonance imaging (NMRI).

Although the three aforementioned methods are able to measure the physical conditions of an aortic artery, each has its own drawbacks:

(1) Ultrasound measurements may be accurate and the health examination costs may be low (around 55 US dollars); but due to interference from gastric air, screening is often difficult with obese people. During measurements, it is difficult to interpret the image, and experts and experienced technicians are required to perform the measurements. Moreover, in typical ultrasound examinations, organs positioned near the abdomen are usually examined, but the aortic artery is not specifically examined.

(2) The examination costs of CT and NMRI are high, and these measurement methods are time consuming. Moreover, problems such as radiation and allergic reaction to medicine exist; hence, these methods are unsuitable for typical health examination screenings. Furthermore, in various countries, the medical insurance do not cover these two types of screenings, and the expensive examination fees must be paid by the patients themselves. Therefore, these two examination methods are unlikely to be accepted by the general public.

Accordingly, developing a lightweight, simple, and innovative detection device and method would help facilitate the examination for the general public and the clinical risks would be lowered.

### SUMMARY

The disclosure provides an aortic artery measuring probe.

The disclosure provides an aortic artery measuring device.

The disclosure provides a method of measuring a diameter of an aortic artery.

The aortic artery measuring probe includes a flexible substrate and a sensor array disposed on the flexible array. The sensor array has M×N ultra-wideband sensors, in which M is an integer greater than or equal to 1, and N is an integer greater than or equal to 2. The ultra-wideband sensors are positioned on a to-be-measured subject and the flexible substrate is deformed to a profile conforming to the profile of the subject. When N is equal to 2, the ultra-wideband sensors may be manually adjusted to positions in a row direction (e.g. the horizontal direction) and to receive an echo signal of the aortic artery. When the ultra-wideband sensors receive the same echo signal of the aortic artery, an intersecting point of the extended center lines of two ultra-wideband sensors is closest to a center of the aortic artery of the subject. When N is greater than or equal to 3, a diameter of the aortic artery may be defined by three adjacent ultra-wideband sensors. In light of the foregoing, the ultra-wideband sensors of the aortic artery measuring probe may have different dispositions, and there are more flexibility in the preferred examination methods due to the different dispositions.

The aortic artery measuring device measures a diameter of an aortic artery of a subject to be measured. The aortic artery measuring device includes the aortic artery measuring probe and a signal processing module electrically connected to each other. The ultra-wideband sensors in the aortic artery measuring probe transmit a radio wave into the subject, the radio wave is reflected by a tissue interface of an artery wall of the aortic artery of the subject, the ultra-wideband sensors receive a reflected signal formed by the reflection of the radio wave, and the reflected signal is analyzed by the signal processing module to define the diameter of the aortic artery.

The method of measuring the diameter of the aortic artery of a subject includes at least the following steps: providing the aortic artery measuring device; positioning the aortic artery measuring probe of the aortic artery measuring device at a first position of the subject for a first predetermined time interval; within the first predetermined time interval, the ultra-wideband sensors transmitting a radio wave into the subject, the radio wave reflected by a tissue interface of an artery wall of the aortic artery of the subject, and the ultra-wideband sensors receiving a reflected signal formed by the reflection of the radio wave; and the signal processing module analyzing the reflected signal to define the diameter of the aortic artery.

Several exemplary embodiments accompanied with figures are described in detail below to further describe the disclosure in details.

### BRIEF DESCRIPTION OF THE DRAWINGS

The accompanying drawings constituting a part of this specification are incorporated herein to provide a further understanding of the disclosure. Here, the drawings illustrate embodiments of the disclosure and, together with the description, serve to explain the principles of the disclosure.

FIGS. 1 and 2 are schematic views of an aortic artery measuring probe at different angles according to a first embodiment of the disclosure.

FIG. 3 is a schematic view of an ultra-wideband sensor according to the first embodiment of the disclosure.

FIG. 4 is a schematic view of an aortic artery measuring device according to the first embodiment of the disclosure.

FIG. 5 is a schematic view of positioning an aortic artery measuring probe on a subject to perform an examination according to the first embodiment of the disclosure.

FIG. 6 is a flow diagram of a method of measuring a diameter of an aortic artery according to an exemplary embodiment.

FIG. 7 is a schematic view of an operation of an aortic artery measuring probe on a subject for performing an examination according to the first embodiment of the disclosure.

FIG. 8 is a schematic view of an operation of an aortic artery measuring probe on a subject for performing an examination according to another exemplary embodiment.

FIG. 9 is a schematic view of an ultra-wideband sensing probe according to a second embodiment of the disclosure.

FIG. 10 is a schematic view of an aortic artery measuring probe being positioned on a subject for performing an examination according to the second embodiment of the disclosure.

FIG. 11 is a schematic view of an operation of an aortic artery measuring probe on a subject for performing an examination according to the second embodiment of the disclosure.

FIG. 12 is a schematic view of an operation of an aortic artery measuring probe on a subject for performing an examination according to another exemplary embodiment.

### DETAILED DESCRIPTION OF DISCLOSED EMBODIMENTS

Descriptions of the disclosure are given with reference to the exemplary embodiments illustrated with accompanied drawings. Nevertheless, the disclosure may be embodied in many different forms and should not be construed as limited to the embodiments set forth herein. In actuality, the embodiments are provided to render the disclosure more explicit and complete, so as to fully convey the scope of the disclosure to people ordinarily skilled in the art. For the purpose of clarity, the sizes of each layer and element may be exaggerated in the drawings.

Terminologies used in the disclosure such as “first” and “second” used to describe each element, component, and location etc. should not be construed as limiting these elements, components, and locations. These terminologies are merely used to differentiate between one element, component, location, and another element, component, or location. Therefore, without departing from the teachings of the embodiments, the first element, component, or location referred in the disclosure below may also be referred as the second element, component, or location.

To facilitate description, terminology related to space may be used in the disclosure (e.g., “below”, “underneath”, “under”, “above”, “over”) to describe the relationship between one element or structural characteristic and another element or structural characteristic. For a device currently being used or operated, the terminology related to space not only includes the directions indicated by the drawings, but also include different directions. For example, if the element used as the reference point in the drawings is changed, then descriptions such as “under” or “below” may be changed to “over” or “above”. Therefore, the description of the positional relationships between the elements depends on the reference point.

The terminology used in the disclosure is for describing each specific embodiment, and should not be construed as limiting the embodiments. The singular forms used in the disclosure such as “a”, “one”, and “the” should also include their corresponding plural forms, unless otherwise specified in the disclosure. When the disclosure uses terminology such as “include” and/or “comprise”, this represents the designated structural characteristic, entity, step, operation, element, and/or component exists. However, this does not exclude the possibility that one or more other structural characteristic, entity, step, operation, element, component, and/or group exists or may be added.

The disclosure described herein is with reference to the exemplary embodiments illustrated with accompanied drawings, which are schematic views of the ideal implementation (and central structure). Accordingly, the variations of the shapes in the drawings due to, for example, manufacturing techniques and/or tolerances should be within expectations.

According to statistical data, when the aortic diameter is greater than 5.5 cm, the subject is in the high risk group of aortic artery rupture or dissection. Moreover, when an aortic aneurysm ruptures, the survival rate is only 18% even when hospital care is provided. For many patients who died because of the rupture or dissection of the aortic artery, the cause of death is typically categorized as a stroke. If early detection is possible, the risk of death can be lowered with the assistance of artery surgery and vascular stent implantation. However, since the growth of the aortic aneurysm does not exhibit any characteristic or symptom which can be observed externally, it is usually not possible for early discovery by the patient to take precaution and make an immediate response. Therefore, health surveillance devices for measuring aortic diameter become highly valuable.

### First Embodiment

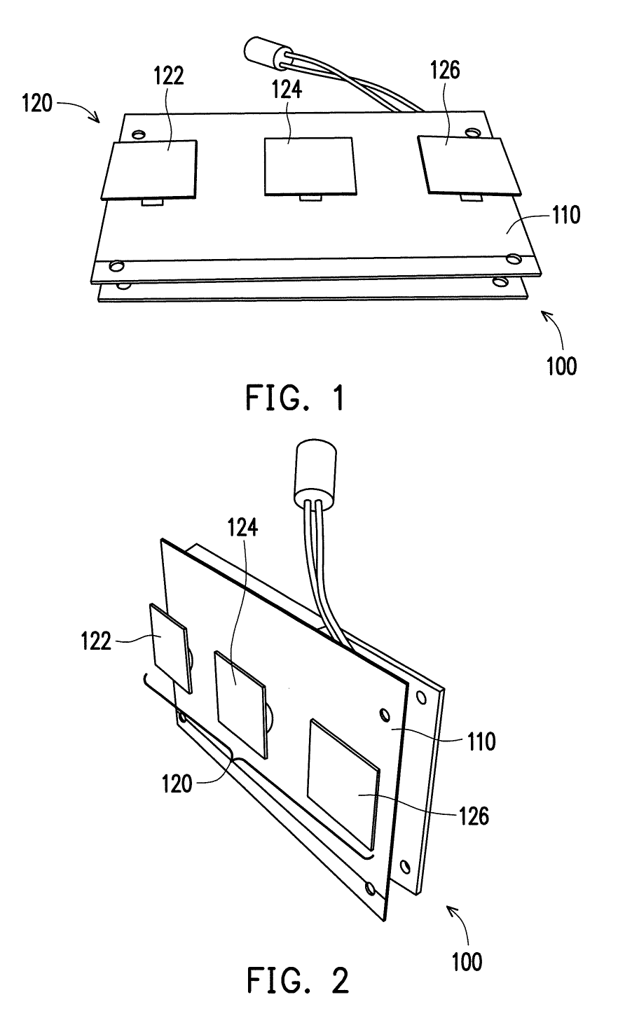

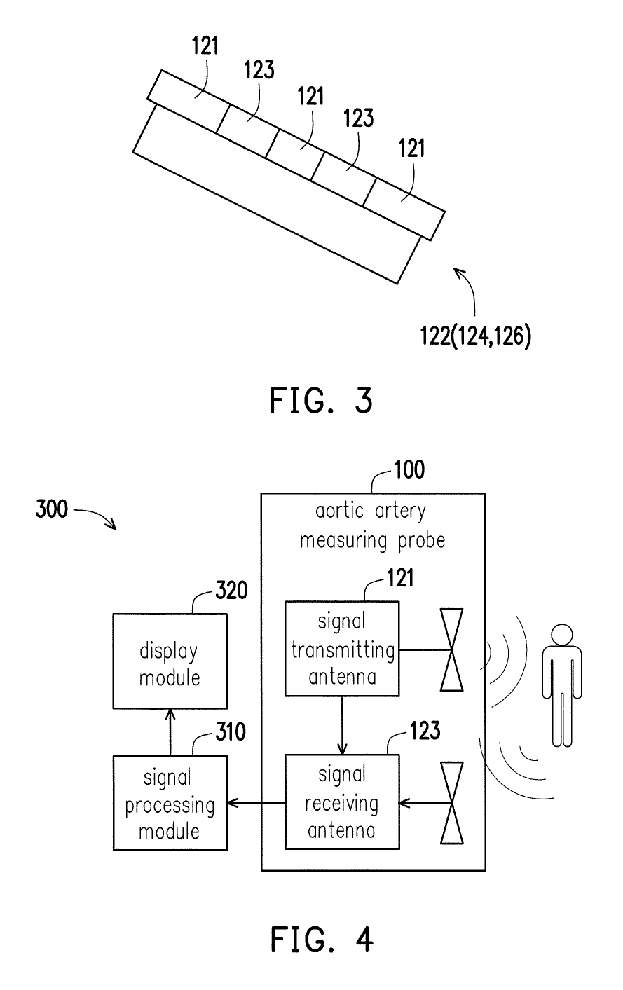

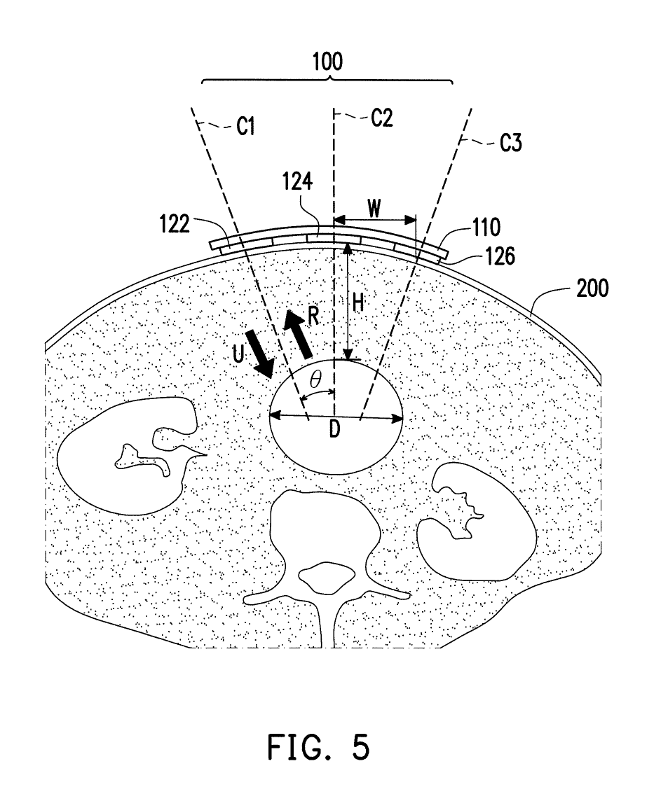

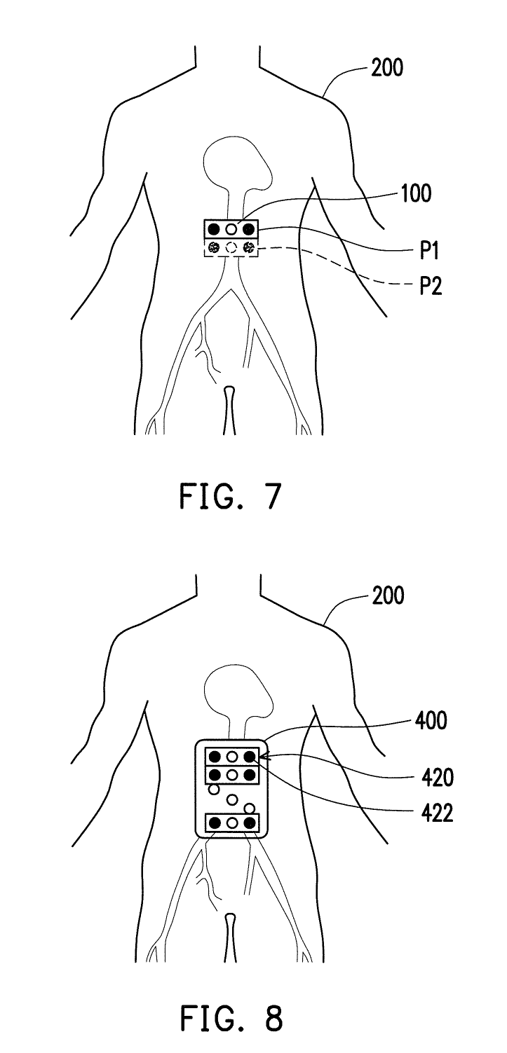

FIGS. 1 and 2 are schematic views at different angles of an aortic artery measuring probe according to a first embodiment of the disclosure, and FIG. 3 is a schematic view of an ultra-wideband sensor. With reference to FIGS. 1, 2, and 3, the disclosure provides an aortic artery measuring probe 100, including a flexible substrate 110 and a sensor array 120 disposed on the flexible array 110. The sensor array 120 has M×N ultra-wideband sensors, and in the present embodiment, M is 1 and N is 3. The ultra-wideband sensors 122, 124, and 126 are positioned on a to-be-measured subject 200 (as shown in FIG. 7). In this exemplary embodiment, the ultra-wideband sensors 122, 124, and 126 are positioned on the to-be-measured subject 200 and focused on the target aortic artery, after the flexible substrate is deformed into an arc and focused on the aortic artery of the subject. In brief, the aortic artery measuring probe 100 of the present embodiment is formed with a flexible substrate 110 and three ultra-wideband sensors 122, 124, and 126 arranged in a row, and the aortic artery measuring probe 100 is applied in measuring a diameter D of an aortic artery (labeled in FIG. 5).

More specifically, each of the ultra-wideband sensors 122, 124, and 126 in the aortic artery measuring probe 100 includes at least one signal transmitting antenna transmitting a radio wave U and at least one signal receiving antenna 123 receiving a reflected signal R. The signal transmitting antenna 121 and the signal receiving antenna 123 may be alternately arranged, although the disclosure is not limited to this arrangement method, and the arrangement method of the signal transmitting antenna 121 and the signal receiving antenna 123 may be adjusted according to an actual requirement. Moreover, a bandwidth of each of the ultra-wideband sensors 122, 124, and 126 is between 0.5-10 GHz. The flexible substrate 110 may deform with the profile of the subject into an arc, such that the shape of the arrangement of the ultra-wideband sensors 122, 124, and 126 is closer to the shape of the aortic artery (as shown in FIG. 5).

FIG. 4 is a schematic view of an aortic artery measuring device, and FIG. 5 is a schematic view of an aortic artery measuring probe positioned on a subject to perform a measurement. With reference to FIGS. 2, 3, 4, and 5, the aortic artery measuring probe 100 is applied in an aortic artery measuring device 300. The aortic artery measuring probe 100 is electrically connected with a signal processing module 310. Accordingly, when the signal transmitting antenna 121 of the ultra-wideband sensors 122, 124, and 126 transmits the radio wave U into the subject, and the signal receiving antenna 123 receives the reflected signal R, the reflected signal R may be analyzed and processed by the signal processing module 310, so as to further define the diameter D of the aortic artery.

Moreover, the aortic artery measuring device 300 may further include a display module 320 electrically connected with the signal processing module 310. The display module 320 displays a cross-sectional model of the aortic artery or related data defined and constructed by the signal processing module 310.

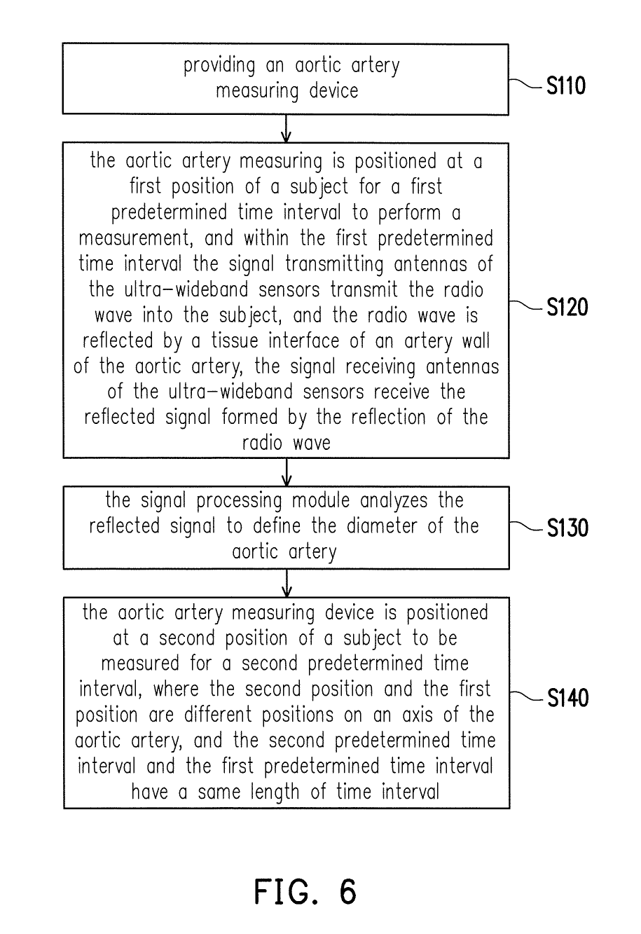

A method of measuring a diameter of an aortic artery by applying the aortic artery measuring device 300 is described below, in which the three ultra-wideband sensors 122, 124, and 126 of the sensor array 120 are arranged in a row. FIG. 6 is a flow diagram of a method of measuring a diameter of an aortic artery according to an exemplary embodiment. With reference to FIGS. 2, 4, 5, and 6, the method of measuring the diameter of the aortic artery includes at least the following steps. In Step S110, the aortic artery measuring device 300 is provided. In Step S120, the aortic artery measuring probe 100 of the aortic artery measuring device 300 is positioned at a first position P1 of a subject for a first predetermined time interval to perform a measurement. Moreover, within the first predetermined time interval, the signal transmitting antennas 121 (labeled in FIG. 3) of the ultra-wideband sensors 122, 124, and 126 transmit the radio wave U into the subject. The radio wave U is reflected by a tissue interface of an artery wall of the aortic artery of the subject. The signal receiving antennas 123 (labeled in FIG. 3) of the ultra-wideband sensors 122, 124, and 126 receive the reflected signal R formed by the reflection of the radio wave U. In Step S130, the signal processing module 310 analyzes the reflected signal R to define the diameter D of the aortic artery.

FIG. 7 is a schematic view of operating an aortic artery measuring probe on a subject to perform an examination. More specifically, the aortic artery measuring probe 100 is positioned at the first position P1, such as the abdomen, of the subject (e.g. a human body) for the first predetermined time interval. The first predetermined time interval may be set in accordance with clinically gathered data. For example, the required first predetermined time interval for the examination may be set according to factors such as age, height, weight, and/or heart beats per minute. Alternatively, the signal processing module 310 may calculate a time interval from the transmission of the radio wave U by the transmitting antenna 121 to the reception of the reflected signal R by the signal receiving antenna 123, and the signal processing module 310 may record the length of time interval.

A method in which the signal processing module 310 analyzes the reflected signal R to define the diameter of the aortic artery includes differentiating the three adjacent ultra-wideband sensors 122, 124, and 126 in the sensor array 120 into a first ultra-wideband sensor 122, a second ultra-wideband sensor 124, and a third ultra-wideband sensor 126. On a cross-section of the aortic artery corresponding to the sensor array 120, a distance between the second ultra-wideband sensor 124 and an artery wall of the aortic artery is H, a distance between a center line C2 of the second ultra-wideband sensor 124 and any one of the center lines C1 or C3 of the first ultra-wideband sensor 122 or the third ultra-wideband sensor 126 is W, and an included angle between the second ultra-wideband sensor 124 and any one of the first ultra-wideband sensor 122 and the third ultra-wideband sensor 126 is θ. An arc included by the center lines C1 and C3 of the first ultra-wideband sensor 122 and the third ultra-wideband sensor 126 may be obtained by the distance H, the distance W, and the included angle θ. Accordingly, the diameter D of the aortic artery can be defined, and the model of the aortic artery can be constructed. In the present embodiment, a range of the distance H is between 10-45 cm typically.

It should be noted that, a dielectric coefficient of an artery wall of the aortic artery and a decay level of the radio wave U may be preset in the signal processing module 310. Therefore, data that is closer to the actual diameter D of the aortic artery may be obtained after signal processing by the signal processing module 310.

By using the aforementioned steps, data regarding the diameter D of the aortic artery of the subject at the first position P1 can be preliminarily obtained. Thereafter, the examiner may compare this data with a predetermined data, so as to determine whether the aortic artery of the subject being measured has an aneurysm according to a comparison result. According to the foregoing description, when the diameter D of the aortic artery is greater than 5.5 cm, the subject being examined by the afore-described method of measuring the diameter of the aortic artery may be suffering from an aneurysm, and the subject belongs in the high risk group of the aortic artery rupture or dissection. Accordingly, data (e.g., a median reference result) for the diameter D of an aortic artery can be obtained by the method of measuring the diameter of the aortic artery in the disclosure, such that the examiner can make a further determination based on the result (data for diameter D) obtained by the method of measuring the diameter of the aortic artery. The predetermined data may be set in the signal processing module 310 or stored separately in a database, and the storage method of the predetermined data may be selected according to a requirement.

It should be noted that the subject is a living body, and the number of heart beats per minute for each subject is different. Therefore, when the examination performed on a single subject is conducted for a longer time period, the aortic artery measuring device 300 may obtain a plurality of data for the diameter D of the aortic artery within the first predetermined time interval; the plurality of data for the diameter D is resulted from the relaxation and dilatation of the aortic artery t during the pumping of the heart for delivering blood. Accordingly, the smallest diameter D of the aortic artery obtained from the measurements may be selected for comparison with the predetermined data.

The method of measuring the diameter of the aortic artery may further include imaging the aortic artery to construct the cross-sectional model of the aortic artery, and displaying the model of the aortic artery by a display module 320 electrically connected with the signal processing module 310. Moreover, while the display module 320 displays the model of the aortic artery, the distance H, the distance W, the included angle θ, and the diameter D of the aortic artery may be concurrently displayed. The model of the aortic artery may be constructed by image reconstruction which is by using a fitting algorithm on the sensing range signals and the time interval of arrival of the signals.

The method of measuring the diameter of the aortic artery may further include Step S140. With reference to FIGS. 4, 5, 6, and 7, the aortic artery measuring probe 100 of the aortic artery measuring device 300 is positioned at a second position P2 of a subject 200 to be measured for a second predetermined time interval. The second position P2 and the first position P1 are different positions on an axis of the aortic artery. However, the second predetermined time interval and the first predetermined time interval have a same length of time interval, and may also be preset by the examiner. Alternatively, the duration of time interval of the recorded first predetermined time interval may be accessed to serve as the second predetermined time interval.

By measuring at the first position P1 and the second position P2, and by setting the first predetermined time interval and the second predetermined time interval to have the same length of time interval, the relaxation and contraction pattern of the aortic artery can be obtained by using the algorithm. Therefore, the aortic artery measuring probe 100 may be used to examine the subject 200 by scanning along the axis of the aortic artery, and to thereby perform the imaging along the axis of the aortic artery.

In brief, the sensor array 120 of the aortic artery measuring probe 100 has 1×3 ultra-wideband sensors 122, 124, and 126 to facilitate description in the foregoing disclosure. Therefore, when the aortic artery measuring probe 100 is used for examination, the aortic artery measuring probe 100 may be positioned on the subject 200 to examine the diameter D of a particular cross-section of the aortic artery. Alternatively, the aortic artery measuring probe 100 may position on the surface of the subject 200 and be moved along the surface of the subject 200 to perform a scan examination, so as to further perform imaging along the axis of the aortic artery.

Moreover, the aforementioned method compares the examined result and the clinically obtained data to make a determination. On the other hand, a personalized comparison may also be performed to determine whether an abrupt change in the diameter D along the axis of the aortic artery has occurred. More specifically, the diameter D defined by the first position P1 and the diameter D defined by the second position P2 are compared, in which a minimum value or a maximum value in the data obtained using the same predetermined time interval is used for comparison.

FIG. 8 is a schematic view of operating an aortic artery measuring probe on a subject to perform an examination according to another exemplary embodiment. With reference to FIG. 8, a difference from the afore-described embodiment is that, a sensor array 420 of an aortic artery measuring probe 400 of the present embodiment has M×N ultra-wideband sensors 422, in which M is an integer greater than or equal to 2, and N is 3, for example, or an integer greater than 3. As shown in FIG. 8, M rows of ultra-wideband sensors 422 are arranged along the axis of the aortic artery. Therefore, the aortic artery measuring probe 400 does not need be moved along the surface of the subject 200 in order to obtain the diameter D (labeled in FIG. 5) of the aortic artery of different cross-sections. In other words, since the sensor array 420 of the aortic artery measuring probe 400 has more rows of the ultra-wideband sensors 422, the detectable range is larger. Therefore, static deployment is sufficient, and so the aortic artery measuring probe 400 does not need to be moved. It should be noted that, the aortic artery measuring probe 400 having more rows of the ultra-wideband sensors 422 may be chosen to be manufactured as a handheld, or may be fixed on a large equipment according to the requirement.

### Second Embodiment

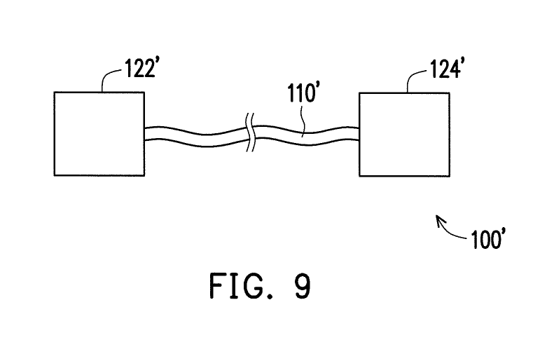

FIG. 9 is a schematic view of an ultra-wideband sensing probe according to a second embodiment of the disclosure. With reference to FIG. 9, the present embodiment is mostly the same as the first embodiment, with a difference in that, the ultra-wideband sensors 122′ and 124′ in an aortic artery measuring probe 100′ of the present embodiment may be arranged in a 1×2 sensor array.

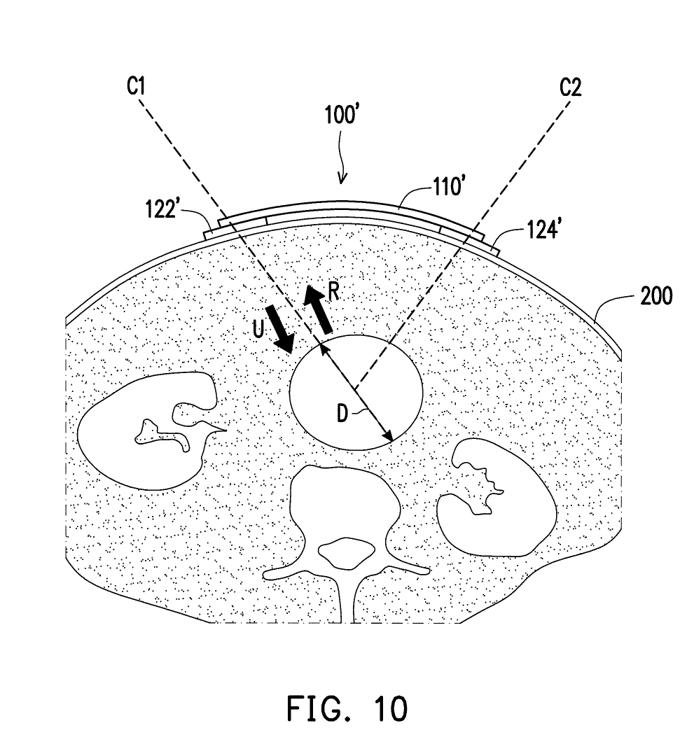

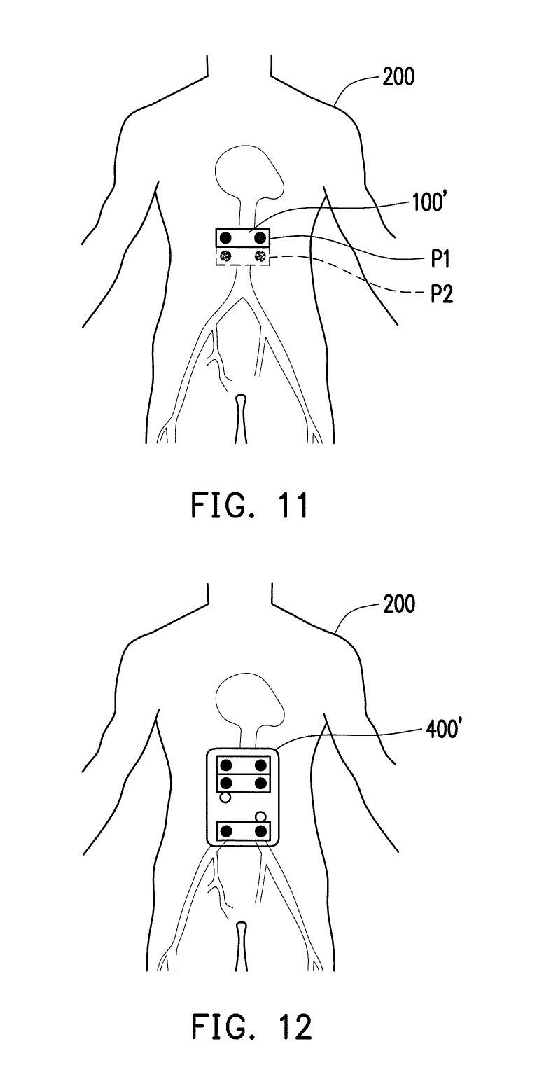

FIG. 10 is a schematic view of positioning an aortic artery measuring probe on a subject to perform an examination according to the second embodiment of the disclosure. With reference to FIGS. 9 and 10, in specifics, when there are only two ultra-wideband sensors in a row of the sensor array in the aortic artery measuring probe 100′, these two ultra-wideband sensors 122′ and 124′ may be disposed on two ends of a flexible substrate 110′ to be connected with each other. A user may also manually adjust the relative positions of ultra-wideband sensors 122′ and 124′ along the row direction (e.g., the row direction being the horizontal direction) and to receive the reflected signal of the aortic artery. When the two ultra-wideband sensors 122′ and 124′ are placed on the subject to performed when both ultra-wideband sensors 122′ and 124′ receive the same reflected signal R of the aortic artery, (the terminology “the same” herein means that the parameters analyzed from the reflected signals are completely the same or extremely similar or close) at this time interval, an intersecting point of the extended center lines C1 and C2 of the two ultra-wideband sensors 122′ and 124′ is closest to a center of the aortic artery of the subject 200. Accordingly, the diameter D of the aortic artery may be further defined. This operating method of the aortic artery measuring probe 100′ may be as shown in FIG. 11, or may be the same as the method of the afore-described first embodiment. The examination may be performed by moving the measuring probe 100′ from the second position P2 up to the first position P1 in a vertical direction along the head to the feet of the subject 200, by moving from the first position P1 down to the second position P2, or by an up-and-down scanning method to examine the subject 200.

With a similar concept, an aortic artery measuring probe 400′ may also be configured with a framework of a sensor array in which M is an integer greater than 1, and N is 2. Since the application of the aortic artery measuring probe 400′ is similar to the methods described in the first embodiment, further elaboration thereof is omitted in the present embodiment.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. An aortic artery measuring probe, comprising:
a flexible substrate;
a sensor array disposed on the flexible substrate having an array of ultra-wideband sensors and the array is a M×N array, wherein M is an integer greater than or equal to 1, and N is an integer greater than or equal to 2, the array of ultra-wideband sensors are positioned on a subject to define a diameter of the aortic artery.

2. The aortic artery measuring probe of claim 1, wherein each ultra-wideband sensor of the array of ultra-wideband sensors comprises:
at least one signal transmitting antenna transmitting a radio wave; and
at least one signal receiving antenna receiving a reflected signal.

3. The aortic artery measuring probe of claim 2, wherein the at least one signal transmitting antenna and the at least one signal receiving antenna are alternatively arranged.

4. The aortic artery measuring probe of claim 1, wherein a bandwidth of each ultra-wideband sensor of the array of ultra-wideband sensors is between 0.5-10 GHz.

5. The aortic artery measuring probe of claim 1, wherein the flexible substrate is deformed into an arc.

6. The aortic artery measuring probe of claim 1, wherein M is 1 and N is 3, and a diameter of the aortic artery of the subject is defined by an included angle between center lines of any two adjacent ultra-wideband sensors arranged in a row, a distance between the center lines of the any two adjacent ultra-wideband sensors on the subject after the flexible substrate is deformed into an arc and focused on the aortic artery of the subject, and a distance between the ultra-wideband sensors and an artery wall of the aortic artery of the subject.

7. The aortic artery measuring probe of claim 1, wherein M is 1 and N is 2, and the ultra-wideband sensors are adjusted to positions in a row direction and to receive a reflected signal of the aortic artery, and when the ultra-wideband sensors receive the same reflected signal of the aortic artery, an intersecting point of the extended center lines of the ultra-wideband sensors is closest to a center of the aortic artery of the subject.

8. An aortic artery measuring device adapted for measuring a diameter of an aortic artery of a subject to be measured, the aortic artery measuring device comprising:
an aortic artery measuring probe, comprising:
a flexible substrate;
a sensor array disposed on the flexible substrate, the sensor array comprising an array of ultra-wideband sensors and the array being a M×N array, wherein M is an integer greater than or equal to 1, and N is an integer greater than or equal to 2, the array of ultra-wideband sensors are positioned on the subject, and the flexible substrate is deformed into an arc and focused on the aortic artery of the subject; and
a signal processing module electrically connected with the aortic artery measuring probe,
the array of ultra-wideband sensors transmitting a radio wave into the subject, the radio wave reflected by a tissue interface of an artery wall of the aortic artery of the subject, the array of ultra-wideband sensors receiving a reflected signal formed by the reflection of the radio wave, and the reflected signal analyzed by the signal processing module to define a diameter of the aortic artery.

9. The aortic artery measuring device of claim 8, wherein each ultra-wideband sensor of the array of ultra-wideband sensors comprises:
at least one signal transmitting antenna transmitting the radio wave; and
at least one signal receiving antenna receiving the reflected signal.

10. The aortic artery measuring device of claim 9, wherein the at least one signal transmitting antenna and the at least one signal receiving antenna are alternatively arranged.

11. The aortic artery measuring device of claim 8, wherein a bandwidth of each ultra-wideband sensor of the array of ultra-wideband sensors is between 0.5-10 GHz.

12. The aortic artery measuring device of claim 8, wherein the flexible substrate is deformed into an arc.

13. The aortic artery measuring device of claim 8, wherein M is 1 and N is 3, and a diameter of the aortic artery of the subject is defined by an included angle between center lines of any two adjacent ultra-wideband sensors of the array of ultra-wideband sensor arranged in a row, a distance after the flexible substrate is deformed into an arc and focused on the aortic artery of the subject between the center lines of the any two adjacent ultra-wideband sensors on the subject, and a distance between the array of ultra-wideband sensors and an artery wall of the aortic artery of the subject.

14. The aortic artery measuring device of claim 8, wherein M is 1 and N is 2, the ultra-wideband sensors being adjusted to positions in a row direction and to receive a reflected signal of the aortic artery, and when the ultra-wideband sensors receive the same reflected signal of the aortic artery, an intersecting point of the extended center lines of the ultra-wideband sensors is closest to a center of the aortic artery of the subject.

15. The aortic artery measuring device of claim 8, further comprising a display module electrically connected with the signal processing module for displaying a model constructed of the aortic artery.

16. A method of measuring a diameter of an aortic artery, comprising:
providing an aortic artery measuring device comprising an aortic artery measuring probe and a signal processing module electrically connected with each other, the aortic artery measuring probe comprising a flexible substrate and a sensor array disposed on the flexible substrate, the sensory array having an array of ultra-wideband sensors and the array being a M×N array, wherein M is an integer greater than or equal to 1, and N is an integer greater than or equal to 2, the array of ultra-wideband sensors are positioned on a subject, and the flexible substrate is deformed into an arc and focused on the aortic artery of the subject;
positioning the aortic artery measuring probe of the aortic artery measuring device at a first position of the subject for a first predetermined time interval;
within the first predetermined time interval, the ultra-wideband sensors transmitting a radio wave into the subject, the radio wave reflected by a tissue interface of an artery wall of the aortic artery of the subject, and the ultra-wideband sensors receiving a reflected signal formed by the reflection of the radio wave; and
the signal processing module analyzing the reflected signal to define a diameter of the aortic artery.

17. The method of measuring the diameter of the aortic artery of claim 16, wherein N is an integer greater than or equal to 3, and the step of the signal processing module analyzing the reflected signal to define the diameter of the aortic artery comprises:
selecting an N−1th ultra-wideband sensor, an Nth ultra-wideband sensor, and an N+1th ultra-wideband sensor adjacent with each other located on an Mth row, and on a cross-section of the aortic artery corresponding to the Mth row of the sensor array, a distance between the Nth ultra-wideband sensor and an artery wall of the aortic artery is H, a distance between a center line of the Nth ultra-wideband sensor and any one of center lines of the N−1 ultra-wideband sensor or the N+1 ultra-wideband sensor is W, and an included angle between the Nth ultra-wideband sensor and any one of the N−1 ultra-wideband sensor and the N+1 ultra-wideband sensor is θ, and the diameter of the aortic artery is defined by the distance H, the distance W, and the included angle θ.

18. The method of measuring the diameter of the aortic artery of claim 17, wherein a range of the distance H is between 10-45 cm.

19. The method of measuring the diameter of the aortic artery of claim 16, wherein the sensor array has 1×2 ultra-wideband sensors, and the ultra-wideband sensors are adjusted to positions in an arranged row direction and to receive a reflected signal of the aortic artery, and when the ultra-wideband sensors receive the same reflected signal of the aortic artery, an intersecting point of the extended center lines of the ultra-wideband sensors is closest to a center of the aortic artery of the subject.

20. The method of measuring the diameter of the aortic artery of claim 16, further comprising positioning the aortic artery measuring probe of the aortic artery measuring device at a second position of the subject for a second predetermined time interval, the first position and the second position are different from each other, and the first predetermined time interval and the second predetermined time interval have a same length of time interval.

21. The method of measuring the diameter of the aortic artery of claim 20, further comprising comparing the diameter of the aortic artery defined at the first position with the diameter of the aortic artery defined at the second position.

22. The method of measuring the diameter of the aortic artery of claim 16, further comprising comparing the diameter of the aortic artery defined at the first position with a predetermined data, wherein the predetermined data is set in the signal processing module or stored in a database.

23. The method of measuring the diameter of the aortic artery of claim 16, wherein the signal processing module presets a dielectric coefficient of an artery wall of the aortic artery and a decay level of the radio wave.

24. The aortic artery measuring device of claim 16, further comprising imaging the aortic artery, and displaying a model constructed of the aortic artery on a display module electrically connected with the signal processing module.
